(19) United States
(12) Patent Application Publication
(10) Pub. No.: US 2009/0117186 A1
(43) Pub. Date: May 7, 2009

(54) TROFOSFAMIDE-CONTAINING FILM-COATED TABLETS AND METHOD FOR THE PRODUCTION THEREOF

(75) Inventor: Berthold Roessler, Halle/Westfalen (DE)

Correspondence Address: BAXTER HEALTHCARE CORPORATION, 1 BAXTER PARKWAY, DF2-2E, DEERFIELD, IL, 60015, US

(73) Assignees: BAXTER INTERNATIONAL INC., Deerfield, IL (US); BAXTER HEALTHCARE S.A., Wallisellen (CH)

(21) Appl. No.: 11/816,391

(22) PCT Filed: Feb. 10, 2006
(86) PCT No.: PCT/EP06/01248

§ 371 Date: May 16, 2008

(30) Foreign Application Priority Data
Feb. 25, 2005 (DE) 10 2005 008 797 3

(51) Int. Cl. A61K 9/36; A61K 9/28; A61K 9/32; A61K 31/66

(52) U.S. Cl. 424/480; 424/474; 424/482; 514/110

(57) ABSTRACT

The invention at hand concerns trofosfamide containing film-coated tablets for oral application and a procedure for their production.

The invention at hand concerns trofosfamide containing film-coated tablets for oral application and procedure for its production.

Trofosfamide (3-(2-chlorethyl)-2-[bis-2-chlorethyl)-amino]-tetrahydro-2H-1,3,2-oxazaphosphorine-2-oxide) is a prodrug which is metabolized in the body into the active forms of the alkylating zytostatica ifosfamide and cyclofosfamide. During the metabolization of trofosfamide, decomposition products are produced at the rate of 6:1 (Ifosfamide:Cyclofosfamide).

Both decomposition products of trofosfamide are substances that are well known and sufficiently proven in drug therapy of tumor diseases. Currently, the cytotoxic agent trofosfamide is used in drug therapy of lymphoreticular tumors and hemoblastoses. For this purpose, in preservation therapy, it is administered daily in an oral dosage of between 50 mg and up to 150 mg.

Trofosfamide (“Ixoten”) is used especially in treating patients at home. This requires that the surroundings of the patient are particularly protected against a contamination through cytotoxic agents. For this purpose, an oral pharmaceutical form of Trofosfamide has to be coated or film-coated in an appropriate way so that agents are not released uncontrollably from the packaging.

Customary methods of coating or film-coating are used in the production of coated tablets, sugar-coated or film-coated tablets [see, for example, Voigt, Pharmazeutische Technologie]. The kind of coating depends on the characteristics of the agent and its responsiveness to production procedures of the respective pharmaceutical form. Hydrolysis susceptible agents, such as trofosfamide, can usually not be sugar-coated (using warm, watery sugar syrup) or coated with watery film suspensions since the agent disintegrates.

Therefore, because of the hydrolysis susceptibility of trofosfamide, currently only one pharmaceutical form of a coated tablet is known in which the agent-containing nucleus is surrounded by a coating of pelleting excipients. Technically, this is realized by, first of all, compressing the tablet nucleus with few excipients on a tablet-producing machine. Subsequently, the nucleus is compressed again, surrounded by a bed of compressed excipients. As a result of this “dry coating,” the nucleus is completely surrounded by an excipients coating. This excipients coating protects the hydrolysis-susceptible agent against moisture, prevents decomposition and, at the same time, protects the patient and his surroundings against contamination of the highly potent agent. Because of the hydrolysis susceptibility of the agent, the production of trofosfamide containing film-coated tablets has not been described in prior art.

Accordingly, the invention at hand has the objective to provide trofosfamide containing film-coated tablets as well as a procedure for production of such film-coated tablets.

This objective is achieved through the embodiments characterized in the claims.

In particular, a film-coated tablet is provided which has a trofosfamide containing nucleus and a film-coating or coating, at which this coating is available by applying a watery suspension which contains one or several film formers. Preferably, the nucleus of the invention-based film-coated tablet has between app. 20 mg and up to app. 200 mg trofosfamide, more preferably, between app. 50 mg and up to 100 mg, especially preferably app. 50 mg trofosfamide as a pharmaceutically effective agent per tablet. Preferably, the total volume of the invention-based film-coated tablet amounts to between app. 100 mg and 400 mg, more preferably, between app. 100 mg and 200 mg, especially preferably, between 140 and 170 mg, at which the total volume of the film-coated tablet is preferably subject to the proportion of trofosfamide. The proportion of the film-coating preferably ranges between app. 1% and app. 15%, more preferably between app. 2% and app. 10%, especially preferably between app. 3% and app. 6%, corresponding to the total volume of the invention-based film-coated tablet.

The proportion of one or several film formers preferably ranges between app. 30% and app. 70%, corresponding to the total volume of the film-coating. The film former used according to the invention is not subject to any restrictions as long as it is pharmaceutically compatible. In a preferred embodiment, the film former is a synthetic or partially synthetic polymer, examples of which are cellulose derivatives such as hypromellose, carboxymethyl cellulose, hydroxy-ethyl cellulose or hydroxy-methyl cellulose, polyvinyl derivatives such as polyvinyl acetate phthalate or polyvinyl pyrrolidone, poly(methyl)acryl acid derivatives such as Eudragit NE, Eudragit E or Eudragit L30, cellulose phthalate such as hydroxypropyl methyl cellulose phthalate, and shelllack.

According to prior art, the trofosfamide containing nucleus can contain, in the weight components customary for pelleting, known, pharmaceutically compatible, excipients and/or carrier substances, such as customary binding agents, blasting agents and/or anti-blocking agents. According to prior art, the watery suspensions containing one or several film formers for the purpose of producing the film-coating can also contain pharmaceutically compatible excipients such as polyethylenglycole, titanium dioxide, anti-foaming agents (for example, Dimeticon) or tenside (for example, polysorbate).

A further subject matter of the invention concerns a procedure to produce a previously defined film-coated tablet, at which the film-coating or coating or emulsifying of the trofosfamide containing nucleus occurs by spraying in a watery suspension (which contains one or several previously defined film formers) at a temperature of between app. 20° C. and app. 40° C., preferably between app. 22° C. and app. 35° C., especially preferably between app. 24° C. and app. 30° C. Preferably, the spraying flow rate to apply the film suspension can occur in the range of between app. 5 and 25 ml/min, more preferably between app. 10 and app. 20 ml/min per 4.5 kg tablet nuclei.

The film-coating of the trofosfamide containing nucleus or the spraying of the previously defined watery suspension on the nucleus is not restricted to the use of a specific device and can, for instance, be performed in a sugar-coating container. It is also possible, by means of a suitable adjustment to the invention-based procedure, to use other spray-coating procedures known in prior art, as, for example, the turbulent mixing procedure, the Accela-Cota procedure, or the Glatt-coating procedure.

Because of a contact with the watery suspension, it is surprisingly possible to prevent, by means of the invention-based procedure, a hydrolytic decomposition of the agent trofosfamide by maintaining the previously mentioned process parameters. Furthermore, it is especially important to maintain these process parameters or process control based on them, since trofosfamide has a relatively low melting point of 51° C. and tends to sinter at app. 40° C. Therefore, it is not possible to heat the tablet nucleus bed, for instance, in a sugar-coating container in order to quickly dry the water from the film suspension.

In an invention-based embodiment to produce trofosfamide containing film-coated tablets, the tablet nuclei are made with a customary rotary tablet-compressing machine. For this purpose, customary pelleting excipients, such as binding agents, blasting agents and/or anti-blocking agents, are used. The tablet nuclei thus obtained are transferred in a sugar-coating container, at which the nuclei amount depends on the size of the container. Through continuous rotating of the container, the nuclei bed is kept moving, while heated supply air of between app. 20 and app. 30° C., preferably between app. 25 and app. 28° C. is blown into the nuclei bed. If a temperature of between app. 20 and app. 28° C. is reached in the nuclei bed, the watery, one or several film formers containing suspension can be sprayed in by means of a nozzle, which can be done continuously with a flow rate of between 10 and 20 ml/min. The valve used according to the invention, the spraying flow rate and pressure in the sugar-coating container have to be adjusted in known fashion to the amounts of tablet nuclei. Under these conditions, the heated supply air quickly dries the surplus water of the suspensions to be applied (or already applied) to the nucleus, so that the hydrolytically instable agent trofosfamide is exposed to the water for only a short period of time, thus preventing decomposition of the agent.

The following examples serve to further explain the invention at hand without being restrictive. The parameters and compositions mentioned in examples 1 and 3 are based on a scheduled quantity of 30,000 tablet nuclei.

### EXAMPLE 1

### Trofosfamide Film-Coated Tablets with a Cellulose Derivative Containing Film-Coating

A trofosfamide film-coated tablet is composed as follows:

| Substance of content | Amount | |
|---|---|---|
| Tablet nucleus | | |
| Trofosfamide | 50.000 | mg |
| Lactose monohydrate (Lactose D10) | 50.000 | mg |
| Microcrystalline cellulose (Avicel PH 101) | 30.000 | mg |
| Corn starch | 10.000 | mg |
| Talcum | 9.000 | mg |
| Colloidal silicon dioxide (Aerosil 200 V) | 1.000 | mg |
| Σ | 150.000 | mg |
| Film-coating | | |
| Hypromellose (Pharmacoat) | 3.428 | mg |
| Polyethylenglycole 6000 | 1.429 | mg |
| Titan dioxide (Pretiox) | 0.971 | mg |
| Sicovit Yellow 10 E172 | 0.143 | mg |
| Dimeticon (Silfar SE4) | 0.029 | mg |
| Σ | 6.000 | mg |
| Film-coating tablet | | |
| Σ | 156.000 | mg |

The components listed under “tablet nucleus” are being strained and mixed homogeneously. For this purpose, current mixing systems can be used. Subsequently, the powder mixture is compressed to tablets with a total weight of app. 150 mg on a rotary tablet-compressing machine.

The tablet nuclei are transferred in a sugar-coating container and, under constant rotation with hot air, are tempered to app. 25° C. By means of a nozzle, the film-coating suspension with the components listed under “film-coating” is continuously sprayed on the tablet nuclei, at which the hot air simultaneously evaporates the solvent.

The process parameters for a quantity of 30,000 film-coated tablets are mentioned in example 3.

### EXAMPLE 2

### Trofosfamide Film-Coated Tablets with a Polyacrylate Derivative Containing Film-Coating

It is also possible to use substituted polyacrylate, such as Eudragit NE for the production of trofosfamide film-coated tablets. In this case, the composition of the tablet nucleus corresponds unmodified to example 1, only the components of the tablet coating vary.

The composition of each film-coated tablet is listed in the following table:

| Substance of content | Amount | |
|---|---|---|
| Tablet nucleus | | |
| Trofosfamide | 50.000 | mg |
| Lactose monohydrate (Lactose D10) | 50.000 | mg |
| Microcrystalline cellulose (Avicel PH 101) | 30.000 | mg |
| Corn starch | 10.000 | mg |
| Talcum | 9.000 | mg |
| Colloidal silicon dioxide (Aerosil 200 V) | 1.000 | mg |
| Σ | 150.000 | mg |
| Film-coating | | |
| Talcum | 2.103 | mg |
| Eudragit NE30 D | 1.402 | mg |
| Polyethylenglycole 6000 | 0.701 | mg |
| Polysorbate 80 (Tween 80) | 0.140 | mg |
| Titan dioxide (Pretiox) | 1.402 | mg |
| Sicovit Yellow 10 E172 | 0.126 | mg |
| Na-carboxymethyl cellulose (Tylopur C30) | 0.122 | mg |
| Dimeticon (Silfar SE4) | 0.014 | mg |
| Σ | 6.000 | mg |
| Film-coating tablet | | |
| Σ | 156.000 | mg |

The production of film-coated tablets is performed analogous to the description in examples 1 and 3.

### EXAMPLE 3

### Process Parameters for a Quantity of 30,000 Film-Coated Tablets in a Sugar-Coating Container

| Spraying system: | Optima | E |
|---|---|---|
| Sugar-coating container rotary speed: | 6.5 | l/min. |
| Nucleus bed temperature: | 25° | C. |
| Supply air temperature: | 28° | C. |
| Spraying flow: | 10-15 | ml/min. |
| Nozzle: | 0.8 | mm |
| Pressure: | 3.5 | bar |
| Spraying time: | 65 | min. |
| Drying time: | 5 | min. |

### EXAMPLE 4

### Trofosfamide Containing Film-Coated Tablet with Cellulose Derivative Containing Film-Coating and 100 mg Dosage

The production of the film-coated tablet with 100 mg dosage per individual pharmaceutical form is performed analogous to the description in examples 1 and 3. The composition is listed in the following table.

A 100 mg trofosfamide containing film-coated tablet has the following composition:

| Substance of content | Amount | |
|---|---|---|
| Tablet nucleus | | |
| Trofosfamide | 100.000 | mg |
| Lactose monohydrate (Lactose D10) | 20.000 | mg |
| Microcrystalline cellulose (Avicel PH 101) | 15.000 | mg |
| Corn starch | 5.000 | mg |
| Talcum | 9.000 | mg |
| Colloidal silicon dioxide (Aerosil 200 V) | 1.000 | mg |
| Σ | 150.000 | mg |
| Film-coating | | |
| Hypromellose (Pharmacoat) | 3.428 | mg |
| Polyethylenglycole 6000 | 1.429 | mg |
| Titan dioxide (Pretiox) | 0.971 | mg |
| Sicovit Yellow lO E172 | 0.143 | mg |
| Dimeticon (Silfar SE4) | 0.029 | mg |
| Σ | 6.000 | mg |
| Film-coating tablet | | |
| Σ | 156.000 | mg |

### EXAMPLE 5

### Trofosfamide Containing Film-Coated Tablet with Cellulose Derivative Containing Film-Coating and 100 mg Dosage and 200 mg Tablet Compound

A trofosfamide containing film-coated tablet has the following composition:

| Substance of content | Amount | |
|---|---|---|
| Tablet nucleus | | |
| Trofosfamide | 100.000 | mg |
| Lactose monohydrate (Lactose D10) | 50.000 | mg |
| Microcrystalline cellulose (Avicel PH 101) | 30.000 | mg |
| Corn starch | 10.000 | mg |
| Talcum | 9.000 | mg |
| Colloidal silicon dioxide (Aerosil 200 V) | 1.000 | mg |
| Σ | 200.000 | mg |
| Film-coating | | |
| Hypromellose (Pharmacoat) | 5.142 | mg |
| Polyethylenglycole 6000 | 2.144 | mg |
| Titan dioxide (Pretiox) | 1.456 | mg |
| Sicovit Yellow 10 E172 | 0.215 | mg |
| Dimeticon (Silfar SE4) | 0.044 | mg |
| Σ | 9.000 | mg |
| Film-coating tablet | | |
| Σ | 209.000 | mg |

The production of the film-coated tablets is performed analogous to the description in examples 1 and 3.

### EXAMPLE 6

### Storage Stability of a Trofosfamide Containing Tablet of Example 1

For the storage at 25° C. of trofosfamide containing film-coated tablets according to example 1, the following data could be determined:

| | Initially | 3 months | 6 months | 9 months | 12 months |
|---|---|---|---|---|---|
| Content | 100% | 100% | 99% | 99% | 100% |

1. A film-coated tablet comprising a trofosfamide containing nucleus and a coating obtained by applying to the nucleus a watery suspension which contains at least one film former.

2. The film-coated tablet of claim 1, wherein the amount of trofosfamide is between 20 mg and 200 mg per film-coated tablet.

3. The film-coated tablet of claim 1 comprising a total volume between 100 mg and 300 mg.

4. The coated film-coated tablet of claim 1, wherein the proportion of the film-coating ranges between 1% and 15% by total volume of the film-coated tablet.

5. The film-coated tablet of claim 1, wherein the proportion of film formers ranges between 30% and 70% by total volume of the film-coating.

6. The film-coated tablet of claim 1, wherein the film former is selected from the group consisting of cellulose derivatives, polyvinyl derivatives, poly(methyl)acryl acid derivatives and combinations thereof.

7. A method for producing a film-coated tablet comprising a trofosfamide containing nucleus, the method comprising spraying a watery suspension on the trofosfamide containing nucleus at a temperature of between 20° C. and 40° C., the watery suspension comprising at least one film former.

8. The method of claim 7, wherein the spraying flow rate to apply the watery suspension ranges between 5 and 25 ml/min.

9. The method of claim 7, wherein the film-coating is performed in a sugar-coating container or by a process selected from the group consisting of turbulent mixing procedure, Accela-Cota procedure and Glatt-coating procedure.
